(19) United States
(12) Patent Application Publication
(10) Pub. No.: US 2020/0000732 A1
(43) Pub. Date: Jan. 2, 2020

(54) PROBIOTIC COMPOSITIONS AND DOSAGE FORMS, AND METHODS FOR PREPARING AND USING THE SAME

(71) Applicant: DolCas Biotech, LLC, Landing, NJ (US)

(72) Inventor: K. Gnaneshwar Rao, Chester, NJ (US)

(21) Appl. No.: 16/426,409

(22) Filed: May 30, 2019

Related U.S. Application Data
(60) Provisional application No. 62/677,944, filed on May 30, 2018.

(51) Int. Cl. A61K 9/48; A61K 35/741; A61K 47/02; A61K 47/44; A61K 47/46

(52) U.S. Cl. CPC A61K 9/4816; A61K 9/4891; A61K 47/46; A61K 47/02; A61K 47/44; A61K 35/741

(57) ABSTRACT

The invention relates to probiotic dosage forms, probiotic compositions contained within the dosage forms, and compositions used for sealing the probiotic dosage forms, as well as methods for preparing and using the probiotic dosage forms.

### FIELD OF INVENTION

The present invention generally relates to an encapsulation technology that optimizes probiotic resiliency, delivery and long-term stability.

### BACKGROUND OF INVENTION

The physiological benefits of probiotics have been intensively investigated and well characterized since their early 20^{th} century association with bowel health and longevity. Predating their association with health, however, was their use in fermented milk products and other cultured foods as a means of preservation and storage. As the benefits of these benevolent bacteria are increasingly elucidated, the industry is now presented with the paradox of how to best preserve the microorganisms so that their health benefits can be actualized. Accordingly, Favaro-Trindade, et al., (J. Microencapsulation, 2002, 19(4):485-494) detailed that probiotics must remain viable and reach the large intestines to exert their beneficial effects on the host. Understanding the attempts that have been made in this regard provides the context for the present invention.

Freeze-drying has been used to preserve probiotics during storage and production. However, maintenance stability of microorganisms in the post-manufacturing environment is a concern. A vegetative state is imposed on microorganisms by drying. However the vegetative state can be reversed by various environmental factors including the amounts of oxygen, moisture and heat present. If the vegetative state is reversed, then aroused bacteria succumb to their innate growth curve, and set off on an ill-timed path to death. Therefore, as the marketed CFU counts significantly declines, the associated therapeutic benefit also declines.

Gross encapsulation is a widely used form of drug/supplement delivery. A two-piece capsule system wholly encases powdered or liquid forms of drugs or supplements for delivery into the digestive tract via the oral route. Two piece capsules prepared with gelatin or hydroxypropyl methylcellulose (HPMC) have been used for delivering probiotic formulations. HPMC capsules provide generic advantages over gelatin capsules, based solely off the type of material it is composed of. These advantages include being vegetarian, hypoallergenic, unreactive to its contents and lawfully compliant with religious dietary specifications.

The use of HPMC over gelatin capsule delivery additionally benefits probiotics by reducing moisture issues inherent of the gelatin shape. Forming the shape of a gelatin capsule is a water-dependent process, whereby the prepared gelatin capsule will include a water component within the capsule. The solubilized water inherent in the hard gelatin capsule vehicle can become a compromising factor for the stability of some sensitive strains of probiotics that require low moisture. Soukoulis, et al. (Food Chemistry, 2014, 159(100):302-308) reported that water activity (a_{w}) level, as a measure of moisture, is among the extrinsic factors that influence the viability of probiotics in any medium. Water activity levels between 0.11-0.22 were identified by Celik and Sullivan (J. Dairy Sciences, 2013, 96:3506-3516) as “ideal for maintaining post-manufacture stabilities, particularly in sensitive strains”.

Water escaping from the capsule is not only detrimental to susceptible strains of encased probiotics by increasing a_{w}, but as the drawn water escapes from the vehicle itself, the compromised container further exposes the environmentally sensitive probiotics. Nagata (Drug Development & Delivery, 2002, 2:2) reported that even as small as a 10% change in the moisture content of the gelatin capsule can create brittleness and cracking of the vehicle.

HPMC offers benefits in terms of moisture, but it has disadvantages in terms of oxygen. According to Nagata, Drug Development & Delivery, 2002, 2:2) HPMC film is looser than gelatin film and oxygen can permeate more freely through HPMC. In this regard the HPMC vehicle poses a more significant threat than gelatin, because microbes can be particularly susceptible to oxygen as some genera thrive in an oxygen-rich environment and will be prompted to emerge from their dormant condition. Others have tried to overcome the drawback of oxygen permeation in HPMC by methods such as formulating with anaerobic bacterium, like Bifidobacterium spp., instead of using more aerobic bacterium like Lactobacillus spp. or combinations thereof. Other countering mechanisms include the use of oxygen-absorbing desiccant packets in the bottle, formulating with prebiotics or prebiotic oils, or capsule-gap bonding techniques in attempts to preclude oxygen entry or abate its effects.

Furthermore, changes in temperature can also awaken susceptible microorganisms. Low-temperature storage settings partially absorb some of the natural decline in CFU, as tempered microorganisms are able to maintain their preserved, freeze-dried state within a small range. Though refrigeration offers stability benefits, it is not always convenient or possible. Ambient temperature stability offers benefit to the manufacturer and the consumer, as ease of portability affects patient compliance and marketing consistency.

To meet this challenge, some of the industry has begun formulating with probiotics formulations that comprise temperature-resilient soil-based bacteria (SBO) or yeast. These soil or plant-derived microorganisms are not native to the human digestive ecology, and therefore are not permanent residents. They act as primers, creating the environment for the increased growth of resident bacteria, but their action is indirect and unpredictable, as they themselves are not direct sources.

Despite the advantages gleaned from the use of HPMC capsules over gelatin and the various approaches to support the resiliency of probiotics in the capsule application, probiotic formulators still employ an additional fortification system to abate further, unaccounted for declines in the CFU counts. Formulators oversupply the probiotics, sometimes packing in 50 to 100 or more percent of the label's CFU claim in order to meet the label claim after a substantial loss. According to Lee and Salminen, (Handbook of Probiotics and Probiotics, 2009, pg. 59), introducing higher probiotic counts during manufacturing is termed ‘overage’. Overages are further described as an expensive measure that should be minimized by optimizing the viability of the organisms by addressing factors like packaging and controlling for environmental stimuli.

Though significant progress has been made in understanding and in some ways mitigating the susceptibilities of manufactured probiotics, much uncertainty still exists. Even with all the mechanisms previously explored, it is still a very common practice to include label claims associated with ‘time of manufacture’, instead of ‘time of expiration/consumption’ guarantees on CFU counts. ‘Time of manufacture’ guarantees preclude a company's accountability for the inevitable loss of CFUs due to the oxygen, moisture and temperature threats posed in the post-manufacturing phase.

The 2003 CODEX Standard for Fermented Milks extends ‘time of consumption’ minimums of 10⁶ CFU/g for use of probiotics in foods whereby label claims are made. To reach clinical benefit, Van Niel et al. (Pediatrics, 2002, 109:678-684) has even suggested that a person's daily intake should be upwards of 107-109 CFU/g. In the dietary supplement industry, however, no such guidelines exist. Without regulation and with the wide variability found in the post-manufacturing environment, from transport to shelf-display, it is the rare exception to find probiotic formulas guaranteed until the time of expiration. For those that do, either cumbersome refrigeration mandates are obliged, human-derived organisms are forfeited for soil-based ones, or expensive, but necessary overages are applied.

It would be desirable, therefore, to identify a shelf-stable formulation strategy that can maintain the viability of probiotic organisms in a capsule dosage form without the introduction of soil-based organisms or expensive overages. The present invention is directed to overcoming these and other deficiencies in the art.

### SUMMARY OF THE INVENTION

A first aspect of the present invention relates to a probiotic dosage form that is suitable for oral administration. The probiotic dosage form includes a sealed, two piece capsule comprised of plant-based cellulose; a composition within the sealed capsule contains a probiotic organism, a carrier agent having a water activity (a_{w}) level at or below 0.5, and from about 1 to about 4 weight percent of a suspending agent, where the weight percent is based on the total weight of the composition; and an anaerobic inert gas within the sealed capsule. The seal is formed by applying externally of the two piece capsule a liquid composition that includes a plant-based cellulose.

A second aspect of the invention relates to a method of promoting gut health in an animal. This method includes orally administering the probiotic dosage form according to the first aspect of the invention to an animal.

A third aspect of the invention relates to a method of preparing a probiotic dosage form that includes providing a two piece capsule comprised of a plant-based cellulose; introducing into one capsule piece a composition that includes a probiotic organism, a carrier agent having a water activity (a_{w}) level at or below 0.5, and from about 1 to about 4 weight percent of a suspending agent, where the weight percent is based on the total weight of the composition; purging oxygen from the other capsule piece using an anaerobic inert gas; securing the two capsule pieces together such that the thus-formed capsule includes the probiotic composition and the anaerobic inert gas; and applying a liquid composition that includes a plant-based cellulose externally of the two piece capsule to form a seal between the two pieces of the capsule.

A fourth aspect of the present invention relates to a liquid composition for use in sealing plant-based cellulose capsules. According to one embodiment, the liquid composition includes from about 4 to about 9 weight percent of a plant-based cellulose, from about 66 to about 71 weight percent of ethanol, from about 22 to about 27 weight percent of isopropyl alcohol, and up to about 5 weight percent of water, where the weight percent is based on the total weight of the liquid composition.

A fifth aspect of the present invention includes a method of promoting stability of a probiotic composition including a mixture of an anaerobic probiotic organism and an aerobic probiotic organism. The method includes forming a composition that includes a mixture of an anaerobic probiotic organism and an aerobic probiotic organism, a carrier agent having a water activity (a_{w}) level at or below 0.5, and from about 1 to about 4 weight percent of a suspending agent, wherein the weight percent is based on the total weight of the composition; and encapsulating the composition within a sealed two piece capsule including a plant-based cellulose in the presence of an inert anaerobic gas to obtain a probiotic dosage form. The forming and encapsulating steps together promote improved stability of both the anaerobic probiotic organism and the aerobic probiotic organism.

A sixth aspect of the present invention relates to a method of decreasing moisture- and/or oxygen-induced arousal of dormant probiotic organisms. The method includes forming a composition that includes the probiotic organisms, a carrier agent having a water activity (a_{w}) level at or below 0.5, and from about 1 to about 4 weight percent of a suspending agent, where the weight percent is based on the total weight of the composition; and encapsulating the probiotic composition in a capsule including a hygroscopic HPMC composition. The forming and encapsulating steps are carried out in (i) an environment including ambient moisture of less than about 80%, (ii) an environment enriched in an inert anaerobic gas, or (iii) both (i) and (ii).

As used herein, the term “about” when used in connection with a numerical value denotes an interval of accuracy that is ±10% in certain embodiments, ±5% in other embodiments, ±2.5% in still further embodiments, and ±1% in yet another embodiment. This term is used in the context of percentage values, including weight percent and concentration, as well as water activity (a_{w}) levels, time periods, and viscosity.

The present inventor has devised a method for successfully suspending probiotics in a low-moisture carrier for use in HPMC capsules. This process specifically controls for water activity levels, which when too high can disrupt the vegetative state of the bacteria, and when too low inactivates critical sites of metabolism. Either extreme proposes premature bacterial cell death and limited intestinal site inoculation. Silica is blended into the low moisture carrier-probiotics blend to attain the uniformity of the suspension, as well as a clean, smooth visual impact. Said mixture enmeshes the individual bacterial colonies, inherently reducing their exposure to oxygen.

This novel methodology is functionally utilized within a collection of separate, known encapsulation practices, that when used together for the particular application of ‘optimizing probiotic resiliency, delivery and long-term stability’, are additionally innovative. Said capsule technology begins with the moisture impervious characteristics of a HPMC two-piece capsule shell. The capsule body is appreciably filled with the probiotic-carrier-silica suspension. The fluid contents are further protected by an anaerobic inert gas flushing technique that displaces residual oxygen and blankets the contents from the oxygen-pervading nature of the HPMC material. The locked capsule is finally wrapped about its joint with an HPMC-derived banding solution that seals the gapped spacing between the sleeved pieces, further preventing oxygen exposure.

The stability and viability of probiotics is dependent on many environmental factors. Four components of the present invention control for the environmental concerns of oxygen permeability, moisture content and temperature. The stability of low-moisture, carrier-immersed resident commensal bacteria was evidenced by 12-month stability testing (FIGS. 2-4). Resiliency of probiotics in ambient temperatures over this duration supports the issuance of ‘time of expiration’ label claims with a much reduced need for expensive CFU overage fortification.

### BRIEF DESCRIPTION OF THE DRAWINGS

FIG. 1 depicts a process for filling capsules with a probiotic composition to obtain a probiotic dosage form of the invention.

FIG. 2 is a graph comparing the stability of B. longum formulated in the safflower oil-suspension (Example 2A) and its respective controls, control A and control C, in a 12 month stability study.

FIG. 3 is a graph comparing the stability of L. rhamnosus species formulated in the safflower oil-suspension (Example 2A) and its control, control B, in a 12 month stability study.

FIG. 4 is a graph in log format showing the sample to sample comparison of the B. longum formulation in safflower oil-suspension (Example 2A), the L. rhamnosus formulation in safflower oil-suspension (Example 2B), and their combination as in the invention (Example 2).

### DETAILED DESCRIPTION OF THE INVENTION

As discussed more fully below, the present invention relates to probiotic dosage forms, probiotic compositions contained within the dosage forms and compositions used for sealing the probiotic dosage forms, as well as methods for preparing and using the probiotic dosage forms. The foundational feature of the present invention is a probiotic emulsion that provides an imperatively low, water activity (or moisture) level, little to no oxygen interaction, and uniformity for the probiotics it suspends.

The present invention is directed to a probiotic dosage form made of a sealed, two piece capsule comprised of a plant-based cellulose and a composition within the sealed capsule. The composition within the capsule includes a probiotic organism, a carrier agent, and a suspending agent; and an anaerobic inert gas is also present within the sealed capsule. As discussed more fully below, the seal is formed by applying a liquid composition comprising a plant-based cellulose externally of the two piece capsule (and allowing solvent to evaporate such the remaining plant-based cellulose seals the seam between the capsule pieces).

The two piece capsule system, with its seal, desirably affords as much moisture resistance as possible during storage, yet suffices for oral delivery of the payload to the intestinal tract. The plant-based cellulose material that forms the major component of the two piece capsule system is selected from the group consisting of alkyl-substituted cellulose ethers, hydroxyalkyl-substituted cellulose ethers, alkylcelluloses, hydroxyalkylcelluloses, hydroxyalkyl-alkylcelluloses, carboxyalkylcelluloses, carboxyalkyl-alkylcelluloses, and mixtures thereof. More specifically the plant-based cellulose of the two piece capsule is selected from the group consisting of methyl cellulose (MC), ethyl cellulose (EC), hydroxymethylcellulose, hydroxyethyl cellulose, hydroxyethylethyl cellulose, hydroxypropyl cellulose (HPC), hydroxypropylmethyl cellulose (HPMC), hydroxypropylmethyl cellulose phthalate, hydroxyethylmethyl cellulose, hydroxybutylmethylcellulose, cellulose acetylphtalate (CAP), sodium carboxymethyl cellulose, methyl cellulose ether, hydroxyethyl cellulose ether, hydroxypropyl cellulose ether, hydroxyethylmethyl cellulose ether, hydroxyethylethyl cellulose ether, hydroxypropylmethyl cellulose ether, and any mixtures thereof. Of these, hydroxypropylmethyl cellulose (HPMC) is preferred.

In addition to the cellulose derivative, which forms the major component of the capsule shell, the capsule shells may additionally contain a gelling agent, one or more pH-adjusting salts, and optionally water. Any of a variety of gelling agents and pH-adjusting salts can be present. Exemplary gelling agents include, without limitation, carrageenan and gellan gum. Gellan gum has the additional benefit of inherently postponing capsule disintegration until an alkaline environment is reached. For certain pH-sensitive probiotic strains, bypassing the acidity of the stomach may prove additionally beneficial. Exemplary pH-adjusting salts include, without limitation, sodium or potassium acetate.

In one preferred embodiment, clear transparent shell uses 91.5% hypromellose (USP+Ph.Eur), and the shell composition further includes carrageenan, potassium acetate, and water.

Cellulosic capsule shells are commercially available from a number of sources including, without limitation, Quali-V® capsule shells from Qualicaps, Inc. (Whitsett, N.C.), Vcaps® and Vcaps® Plus available from Capsugel (Morristown, N.J.), and VegiCaps® available from Catalent Pharma Solutions (Somerset, N.J.). Any of these or other commercial cellulosic capsule shells can be used in accordance with the present invention.

The composition within the sealed capsule is a mixture of one or more probiotic organisms, the carrier agent having a water activity (a_{w}) level at or below 0.5, the suspending agent, and optionally one or more inert ingredients. In certain embodiments, the compositions consists of a mixture of one or more probiotic organisms, the carrier agent having a water activity (a_{w}) level at or below 0.5, and the suspending agent.

As used herein, a “probiotic” refers to any organism, particularly microorganisms that exert a beneficial effect on the host animal such as increased health or resistance to disease. Probiotic organisms can exhibit one or more of the following non-limiting characteristics: non-pathogenic or non-toxic to the host; are present as viable cells, preferably in large numbers; capable of survival, metabolism, and persistence in the gut environment (e.g., resistance to low pH and gastrointestinal acids and secretions); adherence to epithelial cells, particularly the epithelial cells of the gastrointestinal tract; microbicidal or microbistatic activity or effect toward pathogenic bacteria; anticarcinogenic activity; immune modulation activity, particularly immune enhancement; modulatory activity toward the endogenous flora; enhanced urogenital tract health; antiseptic activity in or around wounds and enhanced would healing; reduction in diarrhea; reduction in allergic reactions; reduction in neonatal necrotizing enterocolitis; reduction in inflammatory bowel disease; and reduction in intestinal permeability.

In one embodiment the probiotic organism is an anaerobic probiotic organism, preferably one or more organisms selected from the genus Bifidobacterium (including but not limited to B. longum, B. infantis, B. breve, B. lactis, B. bifidum, B. adolescentis, B. animalis, B. adolescentis, and B. pseudocatenulatum) and the genus Clostridium (including but not limited to C. butyricum). Other known anaerobic probiotic organisms can also be used alone or in combination with organisms within the genera listed above.

In another embodiment the probiotic organism is an aerobic probiotic organism, preferably one or more organisms selected from the genus Lactobacillus (including but not limited to L. casei, L. acidophilus, L. rhamnosus, L. paracasei, L. johnsonii, L. plantarum, L. reuteri, L. salivarius, L. fermentum, L. cremoris, L. helveticus, L. pentosus, L. buchneri, L. gasseri, L. lactis, L. brevis, L. sakei, L. crispatus, L. bulgaricus, L. delbrueckii, L. paraplantarum, and L. kefir). Other known aerobic probiotic organisms can also be used alone or in combination with organisms within the genus listed above.

In a further embodiment the probiotic organism is a facultative anaerobic probiotic organism, preferably one or more organisms selected from Saccharomyces cerevisiae, Enterococcus faecium, Enterococcus faecalis, Streptococcus thermophiles, Bacillus coagulans, Bacillus natto, Bacillus mesentericus, Bacillus clausii, Lactococcus lactis, Lactococcus cremoris, Pediococcus acidilactici, and Pedicoccus pentosaceus. Other known facultative anaerobic probiotic organisms can also be used alone or in combination with organisms within the genera listed above.

Furthermore, the probiotic organism can be a mixture of any two or more of an anaerobic probiotic organism, an aerobic probiotic organism, and a facultative anaerobic probiotic organism. In certain embodiments, a mixture of three or more, four or more, five or more, six or more, seven or more, eight or more, nine or more, or ten or more anaerobic probiotic organisms, aerobic probiotic organisms, and facultative anaerobic probiotic organisms can be used. Preferably, the anaerobic probiotic organism is selected from the from the genera Bifidobacterium and Clostridium, the aerobic probiotic organism is selected from the genus Lactobacillus, and the facultative anaerobic probiotic organism is selected from the group of Saccharomyces cerevisiae, Enterococcus faecium, Enterococcus faecalis, Streptococcus thermophiles, Bacillus coagulans, Bacillus natto, Bacillus mesentericus, Bacillus clausii, Lactococcus lactis, Lactococcus cremoris, Pediococcus acidilactici, and Pedicoccus pentosaceus.

However, the invention should not be limited to any of the aforementioned microorganisms, as it is a common to the practice of formulating probiotics to combine different resident species to diversify or compound therapeutic action, or to combine resident species with transient species, prebiotics or yeasts to produce metabolic intermediates that fuel the lifecycle of colonized strains.

One exemplary combination includes the anaerobic probiotic organism Bifidobacterium longum and the aerobic probiotic organism Lactobacillus rhamnosus. These are exemplified in the examples.

A second exemplary combination includes the anaerobic probiotic organism Bifidobacterium bifidum and the aerobic probiotic organism Lactobacillus acidophilus.

A third exemplary combination includes the anaerobic probiotic organism Bifidobacterium lactis and the aerobic probiotic organism Lactobacillus rhamnosus. This formulation is particularly desirable for use in promoting women's health.

A fourth exemplary combination includes the anaerobic probiotic organism Bifidobacterium infantis and the aerobic probiotic organism Lactobacillus acidophilus. This formulation is particularly desirable for use in promoting children's health.

A fifth exemplary combination includes the anaerobic probiotic organism Bifidobacterium lactis and the aerobic probiotic organism Lactobacillus casei. This formulation is particularly desirable for use in promoting health in geriatric (elderly) populations.

In the compositions of the present invention, the probiotic organism(s) makes up from about 15 to about 65 weight percent of the composition (based on the total weight), preferably from about 30 to about 40 weight percent of the composition. This converts to a per unit dosage of about 11 billion to about 46 billion colony forming units (CFU), preferably from about 20 billion to about 30 billion CFU. Thus, where combinations of probiotic organisms are used in a single formulation, each probiotic organism can be present in an amount of about 5 to about 40 billion CFU/dose, preferably about 10 to about 35 billion CFU/dose, more preferably about 10 to about 25 billion CFU/dose.

The carrier agent should be suitable for sustaining the limited moisture requirements of latent probiotic colonies, and forms a major part of the matrix of the composition. These carrier agents have low water activity (a_{w}) levels, consistent with findings of Celik and Sullivan, J. Dairy Sciences 96:3506-3516 (2013), which is hereby incorporated by reference in its entirety. By “low a_{w} level” or “low water activity level” it is intended that the carrier has an a_{w} level that is at or below 0.5, and in certain embodiments the a_{w} ranges between 0.01-0.5, 0.01-0.4, 0.05-0.5, or 0.05-0.4.

Exemplary carrier agents include, without limitation, safflower oil, olive oil, sunflower oil, fish oil, krill oil, medium chain triglycerides, soy lecithin, sunflower lecithin, shea butter, and any combinations thereof. One preferred carrier agent is safflower oil.

The carrier agent makes up from about 36 to about 66 weight percent of the probiotic composition, preferably from about 46 to about 54 weight percent.

The suspending agent forms a minor part of the matrix of the composition, and is used primarily to control viscosity, keep the probiotics in suspension, and create uniformity in the appearance of the finished product.

Exemplary suspending agents include, without limitation, silica, beeswax, sunflower lecithin, soy lecithin, and mixtures thereof. Of these, silica is preferred, because it porous nature provides significant surface area that aptly binds liquids and scavenges moisture. Silica also controls viscosity, keeps the probiotics in suspensions, and creates uniformity in the finished product. If lecithin is the carrier, then silica is the preferred suspending agent. If lecithin is used as a suspending agent, then the suspending agent includes a mixture of lecithin and silica with lecithin being a major component and silica being a minor component.

Where a mixture of lecithin and silica is used as a suspending agent, then the carrier agent is preferably medium chain triglycerides (MCTs), safflower oil, or a mixture thereof.

The suspending agent is present in an amount of about 0.1 to about 5 weight percent (based on the total weight of the composition), preferably between about 1 percent and about 4 weight percent.

The composition containing the probiotic organisms, carrier, and suspending agent is preferably formed by first mixing together the carrier and suspending agent, and thereafter mixing the probiotic organisms until the final composition is achieved. In general, the initial mixing is carried out at speeds of 50 to about 240 rpm, in an appropriately sized mixing vessel, until the suspending agent is thoroughly mixed with the carrier. In the latter mixing step, much gentler conditions are used to mix the probiotic organisms into the carrier and suspending agent mixture, including mixing speeds below 40 rpm until the composition is uniform.

Both of these mixing steps are preferably conducted at, e.g., 14-20° C., and in an environment-enriched in a suitable anaerobic inert gas (see below) such that little or no oxygen is introduced into the composition during the mixing steps. In addition, it is preferred that these mixing steps are conducted in a humidity-controlled environment as well. According to one embodiment, the mixing steps are carried out in an environment where the air water vapor ranges between about 20 to about 80 percent, more preferably between about 30 to 70 percent.

Ideally, the composition that is ready for encapsulation has a viscosity of between about 100 to about 4000 centipoise at 18° C., more preferably about 400 to about 700 centipoise at 18° C., or even about 500 to about 600 centipoise at 18° C.

Any suitable anaerobic inert gas can be used during the mixing steps described above, as well as to purge air from the body of the capsule, such that the residual gas inside the sealed capsule is primarily the anaerobic inert gas. Exemplary anaerobic inert gases include, without limitation, nitrogen, argon, carbon dioxide, and mixtures thereof. Of these, nitrogen and nitrogen-containing mixtures are preferred. In one embodiment, the anaerobic inert gas contains about 30 to about 90% nitrogen. In another embodiment, the anaerobic inert gas contains about 50 to about 60% nitrogen.

Any of a variety of suitable encapsulation devices can be used to introduce the composition into one half of the capsule shell (e.g., the body), purge oxygen from the other half of the capsule shell (e.g., the cap), and then nest the cap onto the body before the pieces are locked together. Exemplary encapsulation devices include, without limitation, CFS1200™ capsule filling-sealing machine by Capsugel (Lonza; Morristown, N.J.) and LF70™ liquid encapsulation, banding and sealing machine by ACG Pam Pharma Technologies Pvt. Ltd. (Mumbai, India).

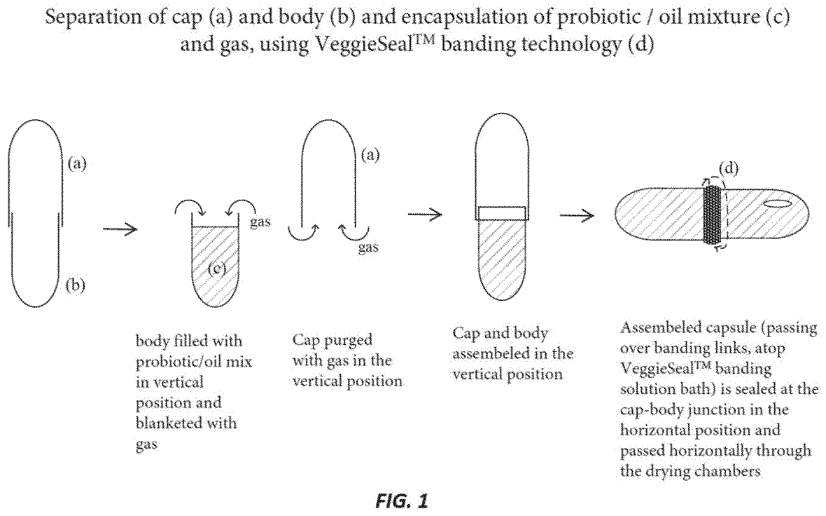

The process of capsule dislodging, filling, and affixing (encapsulating) is illustrated in FIG. 1, in the first three steps. Briefly, empty capsules are loaded into a capsule machine hopper using an automatic empty capsule loader. These capsules are then passed through a chute into a magazine and vertically loaded into capsule bushes where the body of the capsule and caps are separated. The bodies of the capsules are passed under pistons (e.g., ceramic or stainless steel) and the probiotic suspension is carefully pumped into them. The filled capsules are additionally passed under a nitrogen chamber to create a blanket of nitrogen on top of the probiotic mixture. Simultaneously, the caps are flushed with nitrogen to displace oxygen from the caps. (As noted above, other anaerobic inert gas/gases of the type described above can be used.) The caps on top are then brought into contact with the probiotic filled capsule body (located beneath) and compressed to close the capsules. The capsules are then ejected out of the machine through a chute into a filled capsule collection container. The filled capsules can be weighed to confirm that they conform to the appropriate filled weights of the probiotics (fill weight minus the weight of the empty capsule). Approved capsules are then transferred to a banding machine hopper.

Subsequent to encapsulation, the capsules are then sealed using a technique that does not involve heating the capsule shell. This is illustrated in FIG. 1 at the final step, where the capsule is shown in the horizontal position.

Sealing is preferably accomplished using a banding machine, which includes a banding solution bath and mechanisms for delivering the capsules to the bath and applying the banding solution about the joint between the cap and body of the capsules. Exemplary banding machines of this type include CFS1200™ capsule filling-sealing machine by Capsugel (Lonza; Morristown, N.J.) and LF70™ liquid encapsulation, banding and sealing machine by ACG Pam Pharma Technologies Pvt. Ltd. (Mumbai, India).

The liquid banding solution includes alcohol, a plant-based cellulosic material, and optionally water. In one embodiment, the alcohol includes one or more lower alcohols (e.g., ethanol, isopropyl alcohol, propanol, etc.), and the plant-based cellulosic material is the same material used to form the capsule. One exemplary banding solution consists of about 4 to about 9 weight percent of HPMC, about 66 to about 71 weight percent of ethanol, about 22 to about 27 weight percent of isopropyl alcohol, and up to about 5 weight percent of water. Where the weight percent is based on the total weight of the liquid composition.

In certain embodiments, the liquid composition may contain water. In alternative embodiments, the liquid composition can be substantially free of water (i.e., no extraneous water introduced into the formulation). Furthermore the liquid composition may desirably exclude any surfactants.

To facilitate use of the liquid banding solution in the liquid bath of a banding machine, the liquid composition preferably has a viscosity at 18° C. of about 600 to about 1400 centipoise, more preferably about 700 to about 900 centipoise.

Briefly, the prepared banding solution is added to a bath tray and a banding solution reserve container, with a recirculating pump placed between the solution bath and reserve container to ensure an adequate volume of the banding solution in the bath tray at all times. Banding rollers are placed on top of the bath and slid under the banding links (that carry the capsules for the banding machine). After ensuring that the banding solution is properly circulating, the banding rollers can be submersed by ˜2 mm into the banding solution and set to run at a suitable speed such as 30 rpm.

The capsules transferred to the banding machine hooper pass through a magazine and are transferred gently onto the banding links of the machine. As the capsules pass over the banding rollers, the banding solution is gently applied along the joint of the capsule body and cap (i.e., about the entire circumference of the capsule). The now banded, horizontally-oriented capsules are further transferred to carrying links that pass through a drying chamber. Once the capsules are thoroughly dried, they can be placed on drying trays and thereafter inspected for any signs of leakage.

Application of the banding solution externally about the entire circumference of the capsule joint/seam seals the same to minimize the permeation of oxygen and leakage of the capsule liquid contents between the affixed body and cap.

Once the banding solution is thoroughly dried, the capsules can be collected, placed in containers, which are then sealed, and shipped for consumption.

The resulting probiotic dosage form includes a sealed, two piece capsule formed of a plant-based cellulosic material where the seal is also a plant-based cellulosic material, and contained within the capsule are a probiotic composition containing the probiotic organisms, carrier, and suspending agent as well as the anaerobic inert gas.

In one embodiment, the probiotic dosage form includes a sealed, two piece capsule formed where each capsule piece includes a composition of about 91.5 weight percent HPMC, about 2 weight percent carrageenan, about 1.5 weight percent potassium acetate, and about 5 weight percent water (where the weight percent is based on the total weight of the capsule composition); the seal formed externally of the capsule includes HPMC; and the composition within the sealed capsule includes a mixture of Bifidobacterium longum and Lactobacillus rhamnosus, safflower oil, and silica. Also included inside the sealed capsule is nitrogen gas.

Other embodiments, including different combinations of probiotic organism, including those described above, are specifically contemplated.

The invention is further directed to a method of promoting gut health in an animal by orally administering the probiotic dosage to an animal. The oral administration can be carried out once daily, twice daily, or more frequently as desired. Alternatively, oral administration can be carried out less frequently (i.e., periodic administration) as desired.

Yet another aspect of the invention involves a method of promoting stability of a probiotic composition containing a mixture of an anaerobic probiotic organism and an aerobic probiotic organism. The method includes forming a probiotic composition of the type described above (e.g., a mixture of an anaerobic probiotic organism and an aerobic probiotic organism, a carrier agent having a water activity (a_{w}) level at or below 0.5, and from about 1 to about 4 weight percent of a suspending agent), which is then formed into the probiotic dose by encapsulating it in a sealed two piece capsule comprised of a plant-based cellulose in the presence of an inert anaerobic gas. More specifically, the encapsulation includes sealing the two piece capsule with a liquid composition including a plant-based cellulose. It is the forming and encapsulating together that promotes improved stability of both the anaerobic probiotic organism and the aerobic probiotic organism.

In one embodiment of the invention, the improved stability is in comparison to an HPMC capsule lacking a seal applied externally of the HPMC capsule, and also lacking one or more of carrageenan, potassium acetate, and water in the composition forming the HPMC capsule. Additionally, the stability is measured over the course of 12 or more months, such as 15 months, 18 months, 21 months, or 24 or more months.

Another aspect of the invention includes a method of decreasing moisture- and/or oxygen-induced arousal of dormant probiotic organisms. This method includes forming a probiotic composition of the type described above (e.g., a composition of the probiotic organisms, a carrier agent having a water activity (a_{w}) level at or below 0.5, and from about 1 to about 4 weight percent of a suspending agent), which is then encapsulated in a capsule comprising a hygroscopic HPMC composition. The forming and encapsulating are carried out in (i) an environment comprising ambient moisture of less than about 80%, (ii) an environment enriched in an inert anaerobic gas, or (iii) both (i) and (ii), as described above. Preferentially the forming and encapsulating are carried out in an environment with ambient moisture of less than about 80%, preferably less than about 70%, less than about 60%, or between about 20 to about 50%.

Further aspects of the invention will become apparent upon consideration of the following Examples, which illustrate various embodiments for practicing the claimed invention.

### EXAMPLES

The following examples are provided to illustrate embodiments of the present invention but are by no means intended to limit its scope.

### Example 1—Preparation of Banding Solution

A banding solution was prepared using between 4 to 9 weight percent of HPMC, 66 to 71 weight percent ethanol, 22 to 27 weight percent isopropyl alcohol (99%), and water (up to 5 weight percent). Mixing was carried out in a stainless steel mixing vessel.

The HPMC and 99% isopropyl alcohol were added to the container in their respective ratios and stirred well using an impeller at 300 rpm for 10 minutes. When the HPMC was fully dissolved in the isopropyl alcohol, the ethanol was stirred in with the impeller for an additional 10 minutes. Finally, the purified water ration was added into the homogenized solution and stirred for a final 10 minutes.

The resulting solution was used in Example 2 as a banding bath for sealing the capsules.

### Example 2—Preparation of Probiotic Dosage Form

As the ecology of the human digestive tract is quite diverse, it is extremely common to use Bifidobacteria and Lactobacilli of various species formulated together. In the adult digestive tract, the Bifidobacteria and Lactobacilli take up residence in ratio of approximately 10:1, respectively. They are non-competing genera which occupy different parts of the digestive tract. Lactobacilli inoculate the small intestines, whereas Bifidobacteria colonize the colon. Furthermore, the two bacterial types thrive in opposing environments. Lactobacilli flourish best in an oxygen-rich environment, whereas Bifidobacteria prefer an anaerobic abode.

In a first example of the present invention, Bifidobacterium longum were measured out based off a 15 billion colony forming unit/cap amount. Maintaining close fidelity to their natural ratios, 3.1 billion CFU/caps of Lactobacillus rhamnosus was sufficiently measured out to compose a 1,000 capsule batch. The respective quantities of safflower oil and silica were separately weighed out. Under a nitrogen-infused system, the silica was blended into the safflower oil using speeds of 50 to 240 rpm. Once a complete suspension of the silica was attained, the formula-specific probiotics were gently mixed into the oil-silica blend at ambient temperatures between 60-72° F. When the final viscosity of 500 to 550 cps was achieved for the probiotic suspension, it was carefully filled into the HPMC capsule bodies. The probiotic-filled body and respective cap were flushed with a blanket of nitrogen before being overlain and compressed. The locked capsules were ejected from the machine, collected and weighed to confirm proper fill amount. The contents of each individual dosage form are set forth in Table 1 below.

TABLE 1
Dosage Form Composition

| Component Name | Quantity/caps | | Total |
|---|---|---|---|
| B. longum | 1.5 × 10¹⁰ | CFU | |
| L. rhamnosus | 3.1 × 10⁹ | CFU | |
| Safflower oil | 300 | mg | |
| Silica | 6 | mg | |
| | | | 1 capsule |

The approved capsules were filed into magazines from the banding hopper and submersed by 2 mm into the pre-formulated banding solution (from Example 1). The banding rollers applied the banding solution onto the capsule joint, between the telescoped capsule body and cap. The banded capsules were put through a drying chamber and then air dried on trays for an additional 10-12 hours. The process of capsule dislodging, filling, affixing and sealing is illustrated in FIG. 1. The finished product was stored in twist-lid, glass containers at room temperature until needed for testing. Only the quantity required for testing was removed at any one time and the glass bottle re-lidded to secure the remaining product.

### Example 3—Stability Testing of Probiotic Dosage Form

Using the probiotic dosage form of Example 1, real-time stability tests, per the chart below, were conducted over 12 months (at 3 month intervals).

Three controls were included in the stability study and were independently analyzed alongside Example 1 at each time point. Powdered B. longum in hard-shell HPMC capsules (control A) were stored in lidded dark glass bottles. Additionally, powdered L. rhamnosus (control B) and powdered B. longum (control C) as stored in their original packaging in the drum (which is meant to represent a lesser environmentally-labile control).

The stability tests were conducted using well-known serial dilution, plating and enumeration methods. Baseline and quarterly, 1 g or 5 g samples of the probiotic-oil suspension and the respective controls were weighed out into sterilized bottles. A suspension buffer was poured into each bottle to ensure a 100 g sample. The bottles were shaken and the dissolved samples were transferred by pipette into a dilution buffer to create a 10 ml diluted solution. Serial dilutions were completed until the appropriate dilution was prepared. Empty petri dishes were inoculated with the solution in triplicates. After cooling the inoculated plates, a TOS agar plating medium was poured into the dishes and the solution and medium were gently mixed. The dishes were further cooled until their contents were solidified. The dishes were inverted and incubated in an anaerobic environment to allow for a quicker colonization of B. longum.

Although L. rhamnosus colonies grew slowly, visual inspection sufficiently discerned the larger B. longum colonies from the L. rhamnosus. The B. longum colonies were counted and calculated accordingly and designated as Example 2A. Once the viability counts for B. longum were concluded, similar methods were applied to account for L. rhamnosus at each time point. To appropriate for the differences in the bacteria, L. rhamnosus was inoculated on MRS Agar and was placed in an aerobic incubator to potentiate its growth, while stifling the growth of opposing B. longum. L. rhamnosus colonies were counted and calculated accordingly and established as Example 2B. The data is shown below and was plotted accordingly.

Stability tests were performed on the respective controls, as per their genus and the data was analyzed and graphed.

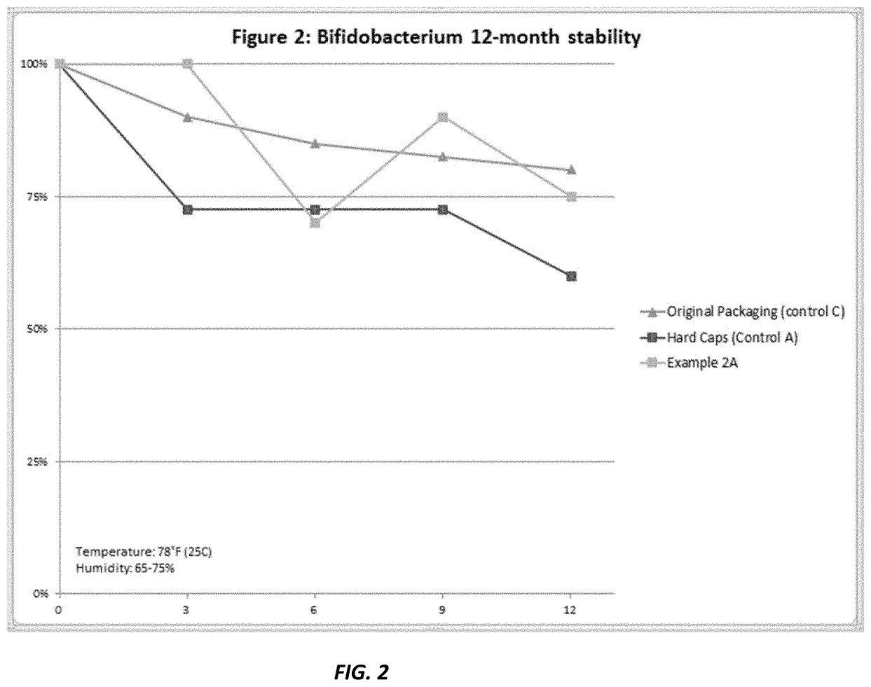

FIG. 2 is a sample to sample comparison of the B. longum formulated in the safflower oil-suspension (Example 2A) and its respective controls, control A and control C.

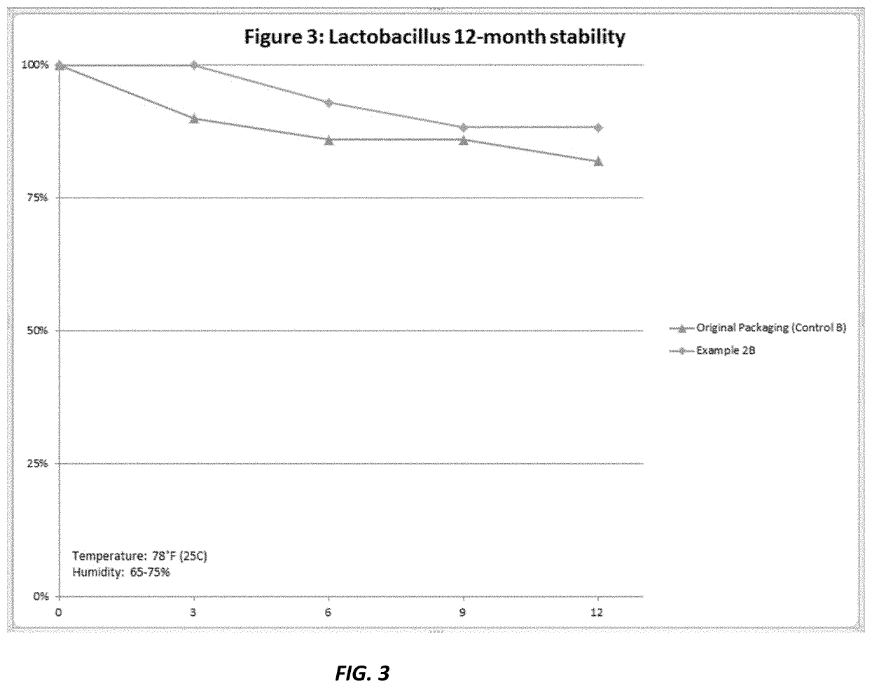

FIG. 3 is a sample to sample comparison of the L. rhamnosus species, as formulated in the safflower oil-suspension (Example 2B) against its respective control, control B.

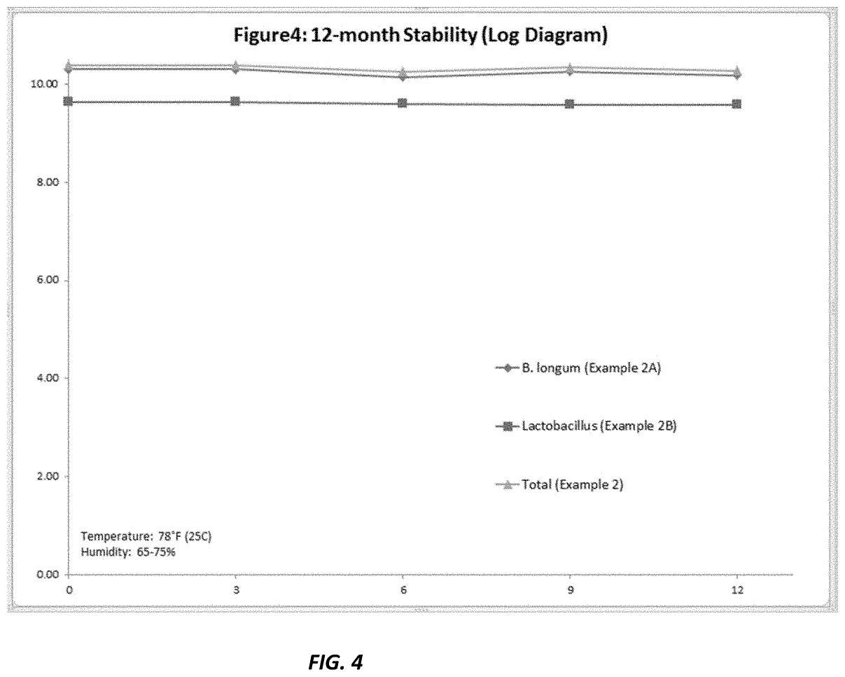

FIG. 4 is a sample to sample comparison in Log format of the B. longum formulated in the safflower oil-suspension (Example 2A), the L. rhamnosus species formulated in the safflower oil-suspension (Example 2B) and their combination as in the invention (Example 2).

Stability data establishes that B. longum (Example 2A), formulated as per the present invention has a 12-month survival of 75% at ambient temperatures. Comparatively, powdered probiotics in conventional hard-shell HPMC capsules (control A) under equivalent conditions only showed 60% survivability. Notably, the 60% survivability of B. longum in a hard shell capsule (control A) had decreased by 20% from its environmentally-protective original packaging (control C). Example 2A showed a more than 15% improvement in survival over the more common finished packaging format (control A), maintaining the survivability of an otherwise moisture and oxygen labile probiotic family (Bifidobacteria) within 5 percent of its original packaging (control C) over the 12 months. To substantiate for such losses over time, it is commonplace for companies to add overage amounts of up to 100-percent, however the present invention reduces the expected overage estimations for hard shell capsule (control A) delivery by 20 percent, from 67% to 33%, as reflected in the chart below.

| Product | | |
|---|---|---|
| (Bifidobacterium longum) | Survival | Formula Overages |
| Example 2A | 75% at 12 months | 33% |
| Control A | 60% at 12 months | 67% |
| (Hard shell capsule) | | |
| Control C | 80% at 12 months | N/A |
| (Original Packaging) | | |

The high survival rate of the probiotics prepared according to the present invention provides the necessary support for enhancing consumer confidence through ‘time to expiration’ guarantees and reduced overage accommodations.

### Prospective Example 4—Preparation and Testing of Alternative Probiotic Dosage Form

Dosage forms of the type described in Example 2 will be prepared using alternative combinations of probiotic organisms, and then tested for 12-month stability using the procedures described in Example 3. The formulations include, in addition to safflower oil (300 mg) and silica (6 mg): (i) between 2.5-3.5×10⁹ CFU Lactobacillus acidophilus and between 1.0-2.5×10¹⁰ Bifidobacterium bifidum; (ii) between 2.5-3.5×10⁹ CFU Lactobacillus rhamnosus and between 1.0-2.5×10¹⁰ Bifidobacterium lactis; (iii) between 2.5-3.5×10⁹ CFU Lactobacillus acidophilus and between 1.0-2.5×10¹⁰ Bifidobacterium infantis; and (iv) between 2.5-3.5×10⁹ CFU Lactobacillus casei and between 1.0-2.5×10¹⁰ Bifidobacterium lactis.

For the 12-month stability testing, each formulation will be tested against 2 comparator arms, one for each organism (i.e. the same organism in its original packaging and in hard shell capsules). Thus, for formulation (i) above, Lactobacillus acidophilus in hard shell capsules and in original capsules as well as Bifidobacterium bifidum in hard shell capsules and in original capsules will serve as controls. The organism count will be taken before and at completion of the stability testing.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

1-8. (canceled)

9. A probiotic dosage form comprising:
a sealed, two piece capsule comprised of a plant-based cellulose,
a composition within the sealed capsule, the composition comprising a probiotic organism, a carrier agent having a water activity (a_{w}) level at or below 0.5, and from about 1 to about 4 weight percent of a suspending agent, wherein the weight percent is based on the total weight of the composition; and
an anaerobic inert gas within the sealed capsule;
wherein the seal is formed by applying a liquid composition comprising a plant-based cellulose externally of the two piece capsule.

10. The probiotic dosage form according to claim 9, wherein the plant-based cellulose of the two piece capsule is the same as the plant-based cellulose that is present within the seal.

11. The probiotic dosage form according to claim 9, wherein the plant-based cellulose of the two piece capsule is different from the plant-based cellulose that is present within the seal.

12. The probiotic dosage form according to claim 9, wherein the plant-based cellulose of the two piece capsule and the plant-based cellulose present within the seal are independently selected from the group consisting of alkyl-substituted cellulose ethers, hydroxyalkyl-substituted cellulose ethers, alkylcelluloses, hydroxyalkylcelluloses, hydroxyalkylalkylcelluloses, carboxyalkylcelluloses, carboxyalkyl-alkylcelluloses, and mixtures thereof.

13. (canceled)

14. The probiotic dosage form according to claim 9, wherein each capsule piece comprises a composition consisting of HPMC, carrageenan, potassium acetate, and water.

15. The probiotic dosage form according to claim 9, wherein the composition within the sealed capsule comprises an anaerobic probiotic organism.

16. (canceled)

17. The probiotic dosage form according to claim 9 wherein the composition within the sealed capsule comprises an aerobic probiotic organism.

18. (canceled)

19. The probiotic dosage form according to claim 9 wherein the composition within the sealed capsule comprises a facultative anaerobic probiotic organism.

20-23. (canceled)

24. The probiotic dosage form according to claim 9, wherein the carrier agent is selected from the group consisting of safflower oil, olive oil, sunflower oil, fish oil, krill oil, medium chain triglycerides, soy lecithin, sunflower lecithin, shea butter, or a combination thereof.

25. The probiotic dosage form according to claim 9, wherein the suspending agent is selected from the group consisting of silica, beeswax, sunflower lecithin, and soy lecithin.

26. The probiotic dosage form according to claim 9, wherein the anaerobic inert gas is nitrogen, argon, or carbon dioxide.

27-29. (canceled)

30. The probiotic dosage form according to claim 9, wherein the composition within the sealed capsule has a viscosity at 18° C. of about 100 to 4000 centipoise.

31. (canceled)

32. A method of promoting gut health in an animal comprising orally administering the probiotic dosage form according to claim 9 to an animal.

33-34. (canceled)

35. A method of preparing a probiotic dosage form comprising:
providing a two piece capsule comprised of a plant-based cellulose;
introducing into one capsule piece a composition comprising a probiotic organism, a carrier agent having a water activity (a_{w}) level at or below 0.5, and from about 1 to about 4 weight percent of a suspending agent, wherein the weight percent is based on the total weight of the composition;
purging oxygen from the other capsule piece using an anaerobic inert gas; and
securing the two capsule pieces together, whereby the capsule comprises therein the composition and the anaerobic inert gas; and
applying a liquid composition comprising a plant-based cellulose externally of the two piece capsule to thereby form a seal between the two pieces of the capsule.

36. The method according to claim 35, wherein said applying is carried out by rolling the capsule, in a horizontal position, through the liquid composition to form a liquid layer band about the circumference of the capsule.

37. The method according to claim 35, wherein said applying is carried out by rolling the capsule, in a horizontal position, through a stream of the liquid composition to form a liquid layer band about the circumference of the capsule.

38-43. (canceled)

44. The method according to claim 35, wherein the composition comprises an anaerobic probiotic organism, an aerobic probiotic organism, a facultative anaerobic probiotic organism, or a mixture of any two or more thereof.

45-46. (canceled)

47. The method according to claim 35, wherein said introducing, purging, and securing is carried out in a low oxygen environment.

48-52. (canceled)

53. The method according to claim 35 further comprising: drying the liquid composition applied externally of the two piece capsule.

54. A method of promoting stability of a probiotic composition containing a mixture of an anaerobic probiotic organism and an aerobic probiotic organism, the method comprising:
forming a composition comprising a mixture of an anaerobic probiotic organism and an aerobic probiotic organism, a carrier agent having a water activity (a_{w}) level at or below 0.5, and from about 1 to about 4 weight percent of a suspending agent, wherein the weight percent is based on the total weight of the composition; and
encapsulating the composition within a sealed two piece capsule comprised of a plant-based cellulose in the presence of an inert anaerobic gas to thereby forming a probiotic dosage form,
wherein said forming and said encapsulating together promote improved stability of both the anaerobic probiotic organism and the aerobic probiotic organism.

55. The method according to claim 54, wherein said encapsulating further comprises sealing the two piece capsule with a liquid composition comprising a plant-based cellulose.

56-59. (canceled)

60. A method of decreasing moisture- and/or oxygen-induced arousal of dormant probiotic organisms, said method comprising the combination of:
forming a composition comprising the probiotic organisms, a carrier agent having a water activity (a_{w}) level at or below 0.5, and from about 1 to about 4 weight percent of a suspending agent, wherein the weight percent is based on the total weight of the composition; and
encapsulating the composition in a capsule comprising a hygroscopic HPMC composition, wherein said forming and said encapsulating are carried out in (i) an environment comprising ambient moisture of less than about 80%, (ii) an environment enriched in an inert anaerobic gas, or (iii) both (i) and (ii).

61. The method according to claim 60, wherein the capsule is a two piece capsule and the method further comprises applying a liquid composition comprising HPMC externally of the two piece capsule to thereby form a seal between the two pieces of the capsule.

62-70. (canceled)
